(19) United States
(12) Patent Application Publication
(10) Pub. No.: US 2012/0219640 A1
(43) Pub. Date: Aug. 30, 2012

(54) Anti-infective solution for athlete's foot

(76) Inventor: KENNETH W. WRIGHT, Los Angeles, CA (US)

(21) Appl. No.: 13/034,976

(22) Filed: Feb. 25, 2011

(51) Int. Cl. A61K 33/18; A61P 31/10

(52) U.S. Cl. 424/667

(57) ABSTRACT

Disclosed herein is a novel use of highly diluted iodine solution in alcohol to effectively treat and prevent athlete's foot skin disease and disinfect and deodorize shoes without staining skin, clothing or surrounding materials.

### BACKGROUND

A widely spread, difficult to manage disease. Athlete's foot (also known as ringworm of the foot and tinea pedis, and also Hong Kong foot is a fungal infection of the skin that causes scaling, flaking, and itch of affected areas. It is caused by fungi in the genus Trichophyton and is typically transmitted in moist areas where people walk barefoot, such as showers or bathhouses. The fungi most commonly associated with athletes foot disease is in the genus Trichophyton, including Trichophyton rubrum, T. mentagrophytes, and Epidermophyton floccosum. Bacteria can also cause a secondary super-infection of the skin contributing to the athlete's foot disease. The fungus grows best in moist skin areas such as between the toes. Although the condition typically affects the feet, it can spread to other areas of the body, including the groin. Athlete's foot can be treated by a number of pharmaceutical and other treatments.

The name “Hong Kong foot” originated from the stationing of the British army in Hong Kong. After the Qing Dynasty of China lost in the First Opium War, they ceded Hong Kong to the United Kingdom. Because the British were used to life in less humid climates of Europe, when they came to Hong Kong, which has a hot and moist climate, they were still wearing their military boots without good ventilation resulting in many British soldiers catching an unknown skin disease with many tiny boils. Some were swollen red with pus, and it was very itchy. Yet, at that time the European physicians had never seen this kind of disease, so they thought it was an epidemic in Hong Kong, so they called it “Hong Kong foot.”

Athlete's foot causes scaling, flaking, and itching of the affected skin. Blisters and cracked skin may also occur, leading to exposed raw tissue, pain, swelling, and inflammation. Secondary bacterial infection can accompany the fungal infection, sometimes requiring a course of oral antibiotics.

The infection can be spread to other areas of the body, such as the groin, and usually is called by a different name once it spreads, such as tinea corporis on the body or limbs and tinea cruris (jock itch or dhobi itch) for an infection of the groin. Tinea pedis most often manifests between the toes, with the space between the fourth and fifth digits most commonly afflicted.

Some individuals may experience an allergic response to the fungus called an “id reaction” in which blisters or vesicles can appear in areas such as the hands, chest and arms. Treatment of the fungus usually results in resolution of the id reaction.

Athlete's foot can usually be diagnosed by visual inspection of the skin, but where the diagnosis is in doubt direct microscopy of a potassium hydroxide preparation (known as a KOH test) may help rule out other possible causes, such as eczema or psoriasis. A KOH preparation is performed on skin scrapings from the affected area. The KOH preparation has an excellent positive predictive value, but occasionally false negative results may be obtained, especially if treatment with an anti-fungal medication has already begun.

If the above diagnoses are inconclusive or if a treatment regimen has already been started, a biopsy of the affected skin (i.e. a sample of the living skin tissue) can be taken for histological examination.

A Wood's lamp, although useful in diagnosing fungal infections of the hair (Tinea capitis), is not usually helpful in diagnosing tinea pedis since the common dermatophytes that cause this disease do not fluoresce under ultraviolet light. However, it can be useful for determining if the disease is due to a non-fungal afflictor.

Athlete's foot is a communicable disease caused by a parasitic fungus in the genus Trichophyton, either Trichophyton rubrum or Trichophyton mentagrophytes. It is typically transmitted in moist environments where people walk barefoot, such as showers, bathhouses, and locker rooms. Sharing footwear with an infected person, or less commonly, by sharing towels with an infected person can also transmit it.

The various parasitic fungi that cause athlete's foot can also cause skin infections on other areas of the body, most often under toenails (onychomycosis) or on the groin (tinea cruris).

The fungi that cause athlete's foot can live on shower floors, wet towels, and footwear, and can spread from person to person from shared contact with showers, towels, etc.

Hygiene, therefore, plays an important role in managing an athlete's foot infection. Since fungi thrive in moist environments, keeping feet and footwear as dry as possible, and avoiding sharing towels, etc., aids prevention of primary infection.

There are many conventional medications (over-the-counter and prescription) as well as alternative treatments for fungal skin infections, including athlete's foot. Important with any treatment plan is the practice of good hygiene. Several placebo controlled studies report that good foot hygiene alone can cure athlete's foot even without medication in 30-40% of the cases. However, placebo-controlled trials of allylamines and azoles for athlete's foot consistently produce much higher percentages of cures than placebo.

Conventional treatment typically involves daily or twice daily application of a topical medication in conjunction with hygiene measures outlined in the above section on prevention. Keeping feet dry and practicing good hygiene is crucial to preventing reinfection. Severe or prolonged fungal skin infections may require treatment with oral anti-fungal medication. Zinc oxide based diaper rash ointment may be used; talcum powder can be used to absorb moisture to kill off the infection.

The fungal infection is often treated with topical antifungal agents, which can take the form of a spray, powder, cream, or gel. The most common ingredients in over-the-counter products are miconazole nitrate (2% typical concentration in the United States) and tolnaftate (1% typical in the U.S.). Terbinafine is another common over-the-counter drug. There exist a large number of prescription antifungal drugs, from several different drug families. These include ketaconazole, itraconazole, naftifine, nystatin,

Some topical applications such as carbol fuchsin (also known in the U.S. as Castellani's paint), often used for intertrigo, work well but in small selected areas. This red dye, used in this treatment like many other vital stains, is a fungicide. Nonetheless, good hygiene is important in curing athlete's foot.

The time-line for cure may be long, often 45 days or longer. The recommended course of treatment is to continue to use the topical treatment for four weeks after the symptoms have subsided to ensure that the fungus has been completely eliminated. However, because the itching associated with the infection subsides quickly, patients may not complete the courses of therapy prescribed.

Anti-itch creams are not recommended as they will alleviate the symptoms but will exacerbate the fungus; this is due to the fact that anti-itch creams typically enhance the moisture content of the skin and encourage fungal growth. For the same reason, some drug manufacturers are using a gel instead of a cream for application of topical drugs (for example, naftin and Lamisil). Novartis, maker of Lamisil, claims that a gel penetrates the skin more quickly than cream.

If the fungal invader is not a dermatophyte but yeast, other medications such as fluconazole may be used. Typically fluconazole is used for candidal vaginal infections moniliasis but has been shown to be of benefit for those with cutaneous yeast infections as well. The most common of these infections occur in the web spaces (intertriginous) of the toes and at the base of the fingernail or toenail. The hallmark of these infections is a cherry red color surrounding the lesion and yellow thick pus.

Oral treatment with griseofulvin was begun early in the 1950s. Because of the tendency to cause liver problems and to provoke aplastic anemia the drugs were used cautiously and sparingly. Over time it was found that those problems were due to the size of the crystal in the manufacturing process and microsize and now ultramicrosize crystals are available with few of the original side effects.[citation needed]

For severe cases, the current preferred oral agent in the UK is the more effective terbinafine.

Other prescription oral antifungals include itraconazole and fluconazole.

Tea tree oil may improve the symptoms but does not cure the underlying fungal infection.

Ajoene, a compound found in garlic, is sometimes used to treat athlete's foot.

Long Felt Need for an Athlete's Foot Treatment: There is a long felt need for a practical and efficacious topical treatment of athlete's foot disease that can be prescribed over the counter. The topical treatment of athlete's foot disease has been the based use of topical anti fungal spray, powder, cream, or gel. Common ingredients in over-the-counter athlete foot remedies include miconazole nitrate and tolnaftate. These drugs have had limited success as resistance to treatment and recurrence is common. Cochrane Database Syst Rev. 2007; reported pooled relative risks of failure to cure (RRFC) for topical treatments for fungal infections of the skin and nails of the human foot for skin. The Cochrane study found even the best anti-fungal topical drugs the allylamines had on average a 30% failure to cure. Other anti-fungal topical drugs i.e., azoles, undecenoic acid, and tolnaftate were less effective. Allylamines are much more expensive than other treatments such as azoles. Cochrane Database after comparing treatment options stated “most cost-effective strategy is first to treat with azoles or undecenoic acid and to use allylamines only if that fails”. Thus despite many topical anti-fungal drugs available on the market there is an unsatisfied long felt need for an inexpensive effective treatment for athlete's foot disease. The anti-fungal drugs also fail to treat bacterial super-infection that can often occur in athlete's foot disease.

Use of Iodine Solutions to Treat Athlete's Foot Disease: Iodine based solutions have been described for the treatment for athletes foot disease but iodine solutions have not been used clinically to a significant extent. Canada Health Category IV monograph: Athlete's Foot treatments 1995 Sep. 11 and the United States FDA Monograph for Topical Antifungal Drug Products lists povadone iodine 10% as an over the counter (OTC) approved topical treatment of athlete's foot disease. Iodine based treatment approved by the United States FDA and Canada Health are specifically for povadone iodine with a concentration of 10%. While iodine solutions such as povadone iodine 10% and tincture of iodine 2% are excellent antimicrobial antiseptics against fungus and bacteria and are relatively inexpensive, this iodine based solutions cause severe staining of skin, clothing, or surrounding material. Elemental iodine the active ingredient in povadone iodine 10% and tincture of iodine 2% has an orange brown color that will stain skin, clothes, and surrounding material. The severe staining character of standard iodine solutions such as povadone iodine 10% and tincture of iodine 2% is a major problem that has made their use undesirable or unacceptable by patients and thus prevented their use for the treatment of athlete's foot disease. As a result, iodine solutions are not marketed for the treatment of athlete's foot disease, and have virtually no market share for the treatment of athlete's foot disease.

The teaching of the use of iodine solutions as a treatment of athlete's foot disease are not to be found in any currently cited in major clinical textbooks, on major medical web sites such as WebMD or E-medicine, or cited in the open access Wikipedia web site as a treatment for athlete's foot.

Iodine in doses of 8 ppm (e.g., 8 mg/L) is known for treating clear water for purification with a 10-minute contact time. Iodine solutions at 50 ppm (e.g., 50 mg/L) will kill bacteria with 1-minute contact time. More significantly, while extremely low concentrations of iodine in water are known to have effective antimicrobial activity, these are never cited in the treatment of athlete's foot.

Problems with shoe disinfection. Shoes are commonly colonized with fungus or bacteria that cause shoe odor as well as infections involving the skin and nails of the foot with the skin between the toes most affected. Moisture in the shoe contributes to fungal and bacterial growth.

Long Felt Need for show infection in Athlete's Foot Treatment: There is a long felt need for a practical and efficacious method for disinfecting shoes. Several products are available to disinfect shoes but most of these remain ineffective because of the time it takes to disinfect the shoe and such products generally cause the staining that makes them undesirable. Use of Iodine alcohol solution as a shoe disinfectant in the instant invention is a surprising application of a very low concentration of iodine in ethanol, which is quick to act, evaporate and leaves no stains. The disinfection of shoes also helps reduce the odor from shoes, a common embarrassing problem in the society.

### DESCRIPTION OF INVENTION

Herein is a description of a novel use of a solution of iodine dissolved in alcohol at a specific low concentration such that the iodine solution has significant antimicrobial activity for the treatment of athlete's foot disease yet it is virtually clear in color so that the solution will not stain skin, surrounding cloths, or surrounding material when applied to the foot and between toes. This iodine solution can be applied topically by spray or absorbent swab to the skin of the foot and specifically between the toes. Iodine is dissolved in a solvent such as isopropyl or ethanol (about 95%). Ethanol and isopropyl alcohol is water-miscible and an antiseptic that when used topically is tolerated well by the skin. This novel ethanol iodine solution has specific characteristics that treat athlete's foot disease including contact antimicrobial activity, drying of the skin of the foot specifically between the toes, and deposition of an imperceptible thin iodine precipitate on and in the skin of the foot and specifically between the toes for long lasting antimicrobial activity.

The concentration of iodine in the instant invention is novel and provides maximum antimicrobial activity yet is low enough not to cause staining of skin, clothes or surrounding material. Iodine's contact antimicrobial activity is directly proportional to the concentration of the iodine. This novel solution is formulated at a concentration of around 50 to 500 mg/liter iodine in a solute of about 95% ethyl or isopropyl alcohol. At this specific iodine concentration around 200 mg/liter iodine in a solute of about 95% ethyl or isopropyl alcohol results in the surprise discovery of no appreciable staining of skin or surrounding materials yet the concentration provides excellent contact antimicrobial activity and a high rate of athletes foot cure. At iodine concentrations around 20-mg/100 mL there is no significant staining of skin, cloths or surrounding materials when sprayed to the foot and toes. This concentration of iodine has antimicrobial activity and leaves a microscopic residue of iodine on the skin.

The high alcohol content (i.e., 95% ethanol, isopropyl alcohol) aspect of the solution dries the skin particularly between the toes and is an important aspect of the treatment of athlete's foot disease. Drying between toes after bathing or showering is otherwise difficult. The solution is specifically a high concentration alcohol 95% that is volatile and is water-miscible. Spray or topical application of the solution results in absorption of moisture from the skin and rapid evaporation of the solution leaves the skin dry. The solution uses high percentage ethanol solvent about 95% that is highly volatile so the solution rapidly evaporates over 3 to 4 minutes leaving the foot and area between the toes dry. An additional action of the high alcohol content solution is its antiseptic working as a contact antibacterial and antifungal.

### EXAMPLE 1

An important action of this novel iodine solution at concentrations about 10 mg/100 mL in ethanol or isopropyl alcohol about 95% is the deposition of an imperceptibly thin precipitate of iodine on and in the skin of the foot specifically between the toes for long lasting antimicrobial activity. Chronic use of the novel solution reduces colonization of the foot by fungus or bacteria preventing athlete's foot disease and reducing recurrent disease. Four patients suffering from various stages of athlete's foot infection were treated successfully by applying a solution of iodine 10 mL/100 mL in isopropyl alcohol.

This novel use of dilute iodine in alcohol treats athlete's foot disease by a combination of mechanisms; contact antimicrobial action, drying skin of the foot and between the toes, and deposition of an imperceptibly thin precipitate of iodine on and in the skin of the foot specifically between the toes for long lasting antimicrobial activity.

### REFERENCES

1. Cochrane Database Syst Rev. 2007; (3):CD001434. Topical treatments for fungal infections of the skin and nails of the foot. Crawford F, Hart R, Bell-Syer S, Torgerson D, Young P, Russell I. Department of Health Sciences & Clinical Evaluation, The University of York, Alciun College, Heslington, York, UK, YO1 5DD. fc5@york.ac.uk

2. Bell-Syer SEM, Hart R, Crawford F, Torgerson D J, Tyrrell W, Russell (22 Apr. 2002). “Oral treatments for fungal infections of the skin of the foot”. Cochrane Database Syst Rev 2 (2): Art. No. CD003584.

3. Category IV Monograph: Athletes Foot treatment 1995 Sep. 11 Health Canada—Drugs and Health Products

4. Memorandum—Department of Health and Human Services, Public Health Service, Food and Drug Administration, Center for Drug Evaluation and Research: Apr. 8, 2004 from Houda Mahayni, Ph.D., Interdisciplinary Scientist Division of Over-the-counter Drug Products, HFD-560. HISTORY AND OVERVIEW OF OTC MONOGRAPH FOR TOPICAL ANTIFUNGAL DRUG PRODUCTS.

5. Rapini, Ronald P.; Bolognia, Jean L.; Jorizzo, Joseph L. (2007). Dermatology: 2-Volume Set. St. Louis: Mosby. pp. 1135. ISBN 1-4160-2999-0.

6. Gupta A K, Skinner A R, Cooper E A (2003). “Interdigital tinea pedis (dermatophytosis simplex and complex) and treatment with ciclopirox 0.77% gel”. Int. J. Dermatol. 42 (Suppl 1): 23-7.

7. Guttman, C; Skinner, Alayne R.; Cooper, Elizabeth A. (2003). “Secondary bacterial infection always accompanies interdigital tinea pedis”. Dermatol Times 4: S12. doi:10.1046/j.1365-4362.42.s1.1.x.

8. Al Hasan M, Fitzgerald SM, Saoudian M, Krishnaswamy G (2004). “Dermatology for the practicing allergist: Tinea pedis and its complications”. Clinical and Molecular Allergy 2 (1): 5. doi:10.1186/1476-7961-2-5.

9. Hainer B L (2003). “Dermatophyte infections”. American Family Physician 67 (1): 101-8.

10. Hirschmann J V, Raugi G J (2000). “Pustular tinea pedis”. J. Am. Acad. Dermatol. 42 (1 Pt 1): 132-3.

11. del Palacio, Amalia; Margarita Garau, Alba Gonzalez-Escalada and M^{a} Teresa Calvo. “Trends in the treatment of dermatophytosis” (PDF). Biology of Dermatophytes and other Keratinophilic Fungi: 148-158.

12. Ajello L, Getz M E (1954). “Recovery of dermatophytes from shoes and a shower stall”. J. Invest. Dermat. 22 (4): 17-22.

13. National Library for Health (6 Sep. 2007). “What is the best treatment for tinea pedis?”. UK National Health Service.

14. Bedinghaus J M, Niedfeldt M W (2001). “Over-the-counter foot remedies”. American Family Physician 64 (5): 791-6.

15. Tong M M, Altman P M, Barnetson R S (1992). “Tea tree oil in the treatment of tinea pedis”. Australasian J. Dermatology 33 (3): 145-9.

16. Satchell A C, Saurajen A, Bell C, Barnetson R S (2002). “Treatment of interdigital tinea pedis with 25% and 50% tea tree oil solution: a randomized, placebo-controlled, blinded study”. Australasian J. Dermatology 43 (3): 175-8.

17. Eliades Ledezma, Katiuska Marcano, Alicia Jorquera, Leonardo De Sousa, Maria Padilla, Mireya Pulgar, Rafael Apitz-Castro (November 2000). “Efficacy of ajoene in the treatment of tinea pedis: A double-blind and comparative study with terbinafine”. J Am Acad Dermatol 43 (5 pt 1): 829-832.

What I claim is:

1. A non-staining composition comprising iodine concentration ranging from 1 mg to 20 mg/100 ml in an organic solvent for use as means of topical treatment of skin conditions caused by infecting organisms and excessive skin moisture in humans and animals in need of such treatment.

2. The composition of claim 1 wherein said organic solvent is selected from a group of solvents comprising ethanol, isopropyl alcohol and acetone.

3. The composition of claim 1 wherein said composition is applied as solution, as spray or using a swab.

4. The composition of claim 1 wherein said concentration of said iodine is between 5 mg/100 mL to 15 mg/100 mL

5. The composition of claim 1 wherein said infecting organism is a fungus.

6. The composition of claim 1 wherein said composition is used to treat athlete's foot disease on a patient in need of said treatment comprising applying said composition to the affected area in an amount and frequency sufficient to ameliorate said athlete's foot.

7. The composition of claim 1 wherein said composition is used in combination with treatment of said athlete's food disease using oral antifungal agents.

8. The composition of claim 1 wherein such composition is used in combination with treatment of said athlete's food disease using other topical antimicrobial and topical antifungal agents.

9. The composition of claim 1 wherein said composition is used to prevent said athlete's foot infection by applying said composition periodically in patients at high risk of acquiring said athlete's foot infection.

10. The composition of claim 1 wherein said composition is used for drying out wet athlete's foot on a patient in need of said treatment comprising applying said composition to the affected area in an amount sufficient to keep the affected area dry and to ameliorate said wet athlete's foot.

11. The composition of claim 1 wherein said composition is used for disinfecting of shoes from fungus and bacteria in need of such disinfection.

12. The composition of claim 1 wherein said composition is used for drying the inside of a shoe to reduce bacterial and fungal colonization of said shoe.

13. The composition of claim 1 wherein said composition is used to reduce shoe odor secondary to fungal and bacterial colonization or infection of foot skin.
